(19) United States
(12) Patent Application Publication
(10) Pub. No.: US 2011/0124605 A1
(43) Pub. Date: May 26, 2011

(54) Use of an S1P Receptor Agonist

(76) Inventors: Shreeram Aradhye, Holzkirchen (DE); Ana de Vera, Basel (CH)

(21) Appl. No.: 12/949,858

(22) Filed: Nov. 19, 2010

Related U.S. Application Data
(60) Provisional application No. 61/263,025, filed on Nov. 20, 2009.

(51) Int. Cl. A61K 31/661; A61K 31/138; A61P 37/00

(52) U.S. Cl. 514/114; 514/654

(57) ABSTRACT

The present invention relates to new uses of S1P receptor modulator or agonist such as fingolimod, for reducing or delaying the progression of cerebral atrophy.

The present invention relates to new uses of S1P receptor modulator or agonist such as fingolimod, for reducing or delaying the progression of cerebral atrophy, e.g. brain atrophy.

In patients with any type of Multiple sclerosis (MS), continuous and irreversible brain volume loss is a consistent finding at all stages of the disease. None of the drugs approved for the treatment of MS have shown consistent atrophy benefits in well-controlled, prospectively planned analyses.

S1P receptor modulators or agonists are compounds which signal as agonists at one or more sphingosine-1 phosphate receptors, e.g. S1P1 to S1P8. Agonist binding to a S1P receptor may e.g. result in dissociation of intracellular heterotrimeric G-proteins into Gα-GTP and Gβγ-GTP, and/or increased phosphorylation of the agonist-occupied receptor and activation of downstream signaling pathways/kinases.

S1P receptor modulators are valuable compounds for the manufacture of medication for the treatment of various conditions in mammals, especially in human beings. For example, efficacy in transplantation has been demonstrated in rats (skin, heart, liver, small bowel), dogs (kidney), and monkeys (kidney) models. Due to their immune-modulating potency, S1P receptor modulators are also useful for the treatment of inflammatory and autoimmune diseases.

Fingolimod (FTY720) is currently being evaluated for the treatment of multiple sclerosis (MS). Evidence indicates that fingolimod acts by preventing lymphocyte egress from lymph nodes. This leads to a reduced infiltration of potentially autoaggressive lymphocytes into the central nervous system (CNS), in particular the number of activated lymphocytes reaching the brain is decreased, resulting in reduced inflammatory destruction. Preclinical evidence also suggests that fingolimod may promote neuroprotective and reparative processes within the CNS via modulation of sphingosine 1-phosphate receptors expressed on neural cells.

FTY720 efficacy in the treatment of multiple sclerosis has been showed in humans (e.g. as described in “FTY720 therapy exerts differential effects on T cell subsets in multiple sclerosis”. Mehling M, et al., Neurology. 2008 Oct. 14; 71(16):1261-7; and “Oral fingolimod (FTY720) for relapsing multiple sclerosis”. Kappos L, Antel J. Comi G, Montalban X, O'Connor P, Polman C H, Haas T, Korn A A, Karlsson G, Radue E W; FTY720 D2201 Study Group, N Engl J. Med. 2006 Sep. 14; 355(11):1124-40.).

S1P receptor modulators or agonists according to the invention are typically sphingosine analogues, such as 2-substituted 2-amino-propane-1,3-diol or 2-amino-propanol derivatives, e.g. a compound comprising a group of formula X

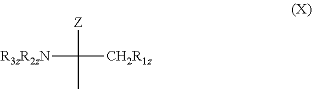

wherein Z is H, C₁₋₆alkyl, C₂₋₆alkenyl, C₂₋₆alkynyl, phenyl, phenyl substituted by OH, C₁₋₆alkyl substituted by 1 to 3 substituents selected from the group consisting of halogen, C₃₋₈cycloalkyl, phenyl and phenyl substituted by OH, or CH₂—R_{4z} wherein R_{4z} is OH, acyloxy or a residue of formula (a)

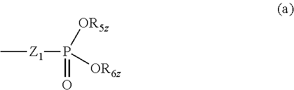

wherein Z₁ is a direct bond or O, preferably O;
each of R_{5z} and R_{6z}, independently, is H, or C₁₋₄alkyl optionally substituted by 1, 2 or 3 halogen atoms;
R_{1z} is OH, acyloxy or a residue of formula (a); and each of R₂, and R₃, independently, is H, C₁₋₄alkyl or acyl.

Group of formula X is a functional group attached as a terminal group to a moiety which may be hydrophilic or lipophilic and comprise one or more aliphatic, alicyclic, aromatic and/or heterocyclic residues, to the extent that the resulting molecule wherein at least one of Z and R_{1z} is or comprises a residue of formula (a), signals as an agonist at one of more sphingosine-1-phosphate receptor.

Examples of appropriate S1P receptor agonists or modulators are, for example:
Compounds as disclosed in EP627406A1, e.g. a compound of formula I

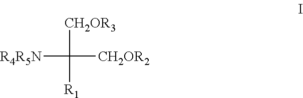

wherein R₁ is a straight- or branched (C₁₂₋₂₂)chain
which may have in the chain a bond or a hetero atom selected from a double bond, a triple bond, O, S, NR₆, wherein R₆ is H, aryl-C₁₋₄alkyl, acyl or (C₁₋₄alkoxy)carbonyl, and carbonyl, and/or
   which may have as a substituent C₁₋₄alkoxy, C₂₋₄alkenyloxy, C₂₋₄alkynyloxy, arylC₁₋₄alkyloxy, acyl, C₁₋₄alkylamino, C₁₋₄alkylthio, acylamino, (C₁₋₄alkoxy)carbonyl, (C₁₋₄alkoxy)-carbonylamino, acyloxy, (C₁₋₄alkyl)carbamoyl, nitro, halogen, amino, hydroxyimino, hydroxy or carboxy; or

### R₁ is

a phenylalkyl wherein alkyl is a straight- or branched (C₆₋₂₀)carbon chain; or
a phenylalkyl wherein alkyl is a straight- or branched (C₁₋₃₀)carbon chain wherein said phenylalkyl is substituted by
a straight- or branched (C₆₋₂₀)carbon chain optionally substituted by halogen,
a straight- or branched (C₆₋₂₀)alkoxy chain optionally substituted by halogen,
a straight- or branched (C₆₋₂₀)alkenyloxy,
phenyl-C₁₋₁₄alkoxy, halophenyl-C₁₋₄alkoxy, phenyl-C₁₋₁₄alkoxy-C₁₋₁₄alkyl, phenoxy-C₁₋₄alkoxy or phenoxy-C₁₋₄alkyl,
cycloalkylalkyl substituted by C₆₋₂₀alkyl,
heteroarylalkyl substituted by C₆₋₂₀alkyl,
heterocyclic C₆₋₂₀alkyl or
heterocyclic alkyl substituted by C₂₋₂₀alkyl,
   and wherein
   the alkyl moiety may have
in the carbon chain, a bond or a heteroatom selected from a double bond, a triple bond, O, S, sulfinyl, sulfonyl, or NR₆, wherein R₆ is as defined above, and
as a substituent C₁₋₄alkoxy, C₂₋₄alkenyloxy, C₂₋₄alkynyloxy, arylC₁₋₄alkyloxy, acyl, C₁₋₄alkyl-amino, C₁₋₄alkylthio, acylamino, (C₁₋₄alkoxy)carbonyl, (C₁₋₄alkoxy)carbonylamino, acyloxy, (C₁₋₄alkyl)carbamoyl, nitro, halogen, amino, hydroxy or carboxy, and
   each of R₂, R₃, R₄ and R₅, independently, is H, C₁₋₄ alkyl or acyl
   or a pharmaceutically acceptable salt or hydrate thereof;
   benzyloxyphenoxy)-2-chlorophenyl]propyl-1,3-propane-diol or 2-amino-2-[4-(benzyloxyphenylthio)-2-chlorophenyl]propyl-1,3-propane-diol,
   or a pharmacological salt, solvate, hydrate or phosphate derivative thereof.

When the compound of formula I has one or more asymmetric centers in the molecule, the present invention is to be understood as embracing the various optical isomers, as well as racemates, diastereoisomers and mixtures thereof are embraced.

The compounds of formula I may exist in free or salt form. Examples of pharmaceutically acceptable salts of the compounds of the formula I include salts with inorganic acids, such as hydrochloride, hydrobromide and sulfate, salts with organic acids, such as acetate, fumarate, maleate, benzoate, citrate, malate, methanesulfonate and benzenesulfonate salts, or, when appropriate, salts with metals such as sodium, potassium, calcium and aluminium, salts with amines, such as triethylamine and salts with dibasic amino acids, such as lysine.

Acyl as indicated above may be a residue R_{y}—CO— wherein R_{y} is C₁₋₆alkyl, C₃₋₆cycloalkyl, phenyl or phenyl-C₁₋₄alkyl. Unless otherwise stated, alkyl, alkoxy, alkenyl or alkynyl may be straight or branched.

Aryl may be phenyl or naphthyl, preferably phenyl.

When in the compounds of formula I the carbon chain as R₁ is substituted, it is preferably substituted by halogen, nitro, amino, hydroxy or carboxy. When the carbon chain is interrupted by an optionally substituted phenylene, the carbon chain is preferably unsubstituted. When the phenylene moiety is substituted, it is preferably substituted by halogen, nitro, amino, methoxy, hydroxy or carboxy.

Preferred compounds of formula I are those wherein R₁ is C₁₃₋₂₀alkyl, optionally substituted by nitro, halogen, amino, hydroxy or carboxy, and, more preferably those wherein R₁ is phenylalkyl substituted by C₆₋₁₄-alkyl chain optionally substituted by halogen and the alkyl moiety is a C₁₋₆alkyl optionally substituted by hydroxy. More preferably, R₁ is phenyl-C₁₋₆alkyl substituted on the phenyl by a straight or branched, preferably straight, C₆₋₁₄alkyl chain. The C₆₋₁₄alkyl chain may be in ortho, meta or para, preferably in para.

Preferably each of R₂ to R₅ is H.

Preferred S1P receptor modulators of the invention are selected 2-amino-2-tetradecyl-1,3-propanediol, pharmacological salt thereof, prodrug, and phosphate thereof. An example of S1P receptor modulator is FTY720, i.e. 2-amino-2-[2-(4-octylphenyl)ethyl]propane-1,3-diol in free form or in a pharmaceutically acceptable salt form (referred to hereinafter as Compound A), e.g. the hydrochloride, as shown:

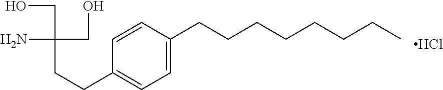

Another preferred S1P receptor modulator is the phosphate derivative form (FTY720-phosphate), referred to hereinafter as Compound B.

In a specific embodiment of the invention, the S1P receptor modulator of the invention, e.g. fingolimod in free form, in a pharmaceutically acceptable salt form or fingolimod-phosphate, is administered orally

As herein above defined, cerebral atrophy, e.g. brain atrophy, refers to the diminution of the size or volume of the brain. It may also refer to the reduction of inflammation in the brain. In a specific embodiment, cerebral atrophy, e.g. brain atrophy, results in a loss of neuros and/or the connections between them.

Atrophy can be generalized, or it can be focal, affecting only a limited area of the brain.

Methods to determine brain atrophy are known to the person skilled in the art. For example, such a determination can be made through imaging the brain or brain tissues of a patient at different time intervals, e.g. by nuclear magnetic resonance (MRI) images. According to the characterization of the tissues that are white matter and grey matter, a computation of the surface area of each tissue can be carried out from MRI slice image.

According to the invention, there is provided:
1.1 The use of a S1P receptor modulator or agonist, e.g. FTY720, a salt or phosphate thereof, e.g. Compound A or Compound B, e.g. hydrochloride salt of FTY720, in the manufacture of a medication, for inhibiting, delaying the progression of or treating brain atrophy.
1.2 The use of a S1P receptor modulator or agonist, e.g. FTY720, a salt or phosphate thereof, in the manufacture of a medication for lessening the loss of brain tissue or reducing brain volume loss.
1.3 The use as defined under 1.1. or 1.2 above wherein the brain atrophy or loss of brain tissue results from an autoimmune disease, e.g. multiple sclerosis.
1.4 The use of FTY720 in the manufacture of a medication as defined under 1.1. to 1.3 above, whereby said medication is administered at a daily dosage of 0.5 mg or 1.25 mg.
2.1 A method for inhibiting brain atrophy, or limiting, reducing the progression of brain atrophy in a subject in need thereof, comprising administering to the subject a S1P receptor modulator or agonist, e.g. FTY720 or a pharmaceutically acceptable salt thereof, or Compound B.
2.2 A method for slowing progression of brain atrophy in a subject in need thereof, comprising administering to the subject a S1P receptor modulator or agonist, e.g. FTY720 or a salt thereof, or Compound B.
2.3 A method as defined under 2.1. or 2.2. above wherein the brain atrophy results from an autoimmune disease, e.g. multiple sclerosis.
2.4. A method as defined under 2.1. or 2.2. above wherein the subject to be treated is affected by an autoimmune disease, e.g. multiple sclerosis.

2.5 A method as defined under 2.1. to 2.4 above, comprising administering to the subject an therapeutically effective amount of a S1P receptor modulator or agonist, e.g. FTY720 or a salt thereof, or Compound B.

2.6 A method as defined under 2.1. to 2.5 above, comprising administering to the subject a daily dosage of FTY720 or a salt thereof, of about 0.5 mg or 1.25 mg, e.g. of about 0.5 mg.

Clinicians usually categorize patients having MS into four types of disease patterns:
Relapsing-remitting (RR-MS): Discrete motor, sensory, cerebellar or visual attacks that occur over 1-2 weeks and often resolve over 1-2 months. Some patients accrue disability with each episode, yet remain clinically stable between relapses. About 85% of patients initially experience the RR form of MS, but within 10 years about half will develop the secondary progressive form.
Secondary-progressive (SP-MS): Initially RR followed by gradually increasing disability, with or without relapses. Major irreversible disabilities appear most often during SP.
Primary-progressive (PP-MS): Progression disease course from onset without any relapses or remissions, affecting about 15% of MS patients.
Progressive-relapsing (PR-MS): Progressive disease from onset with clear acute relapses; periods between relapses characterized by continuing progression.

Accordingly, the patient to be treated with the S1P receptor modulator or agonist, e.g. FTY720, a pharmaceutically acceptable salt thereof, or Compound B, may be affected by one or more of Relapsing-remitting (RRMS), Secondary-progressive (SPMS), Primary-progressive (PPMS) and Progressive-relapsing (PRMS).

According to the invention, multiple sclerosis refers to Relapsing-remitting (RRMS), Secondary-progressive (SPMS), Primary-progressive (PPMS) or Progressive-relapsing (PRMS), for example to RRMS.

Daily dosages required in practicing the method of the present invention when a S1P receptor modulator or agonist is used will vary depending upon, for example, the compound used, the host, the mode of administration and the severity of the condition to be treated. A preferred daily dosage range is about from 0.1 to 100 mg as a single dose or in divided doses. Suitable daily dosages for patients are on the order of from e.g. 0.1 to 50 mg p.o. The S1P receptor modulator or agonist may be administered by any conventional route, in particular enterally, e.g. orally, e.g. in the form of tablets, capsules, drink solutions, nasally, pulmonary (by inhalation) or parenterally, e.g. in the form of injectable solutions or suspensions. In a specific embodiment the S1P receptor modulator or agonist, e.g. FTY720 or a salt thereof, or Compound B, is administered orally. Oral formulation may be in the form of a powder, granule or pellets or a unit dosage form, for example a tablet or capsule. In a specific embodiment, FTY720 or a pharmaceutically acceptable salt thereof is administered in the form of an unit dosage, e.g. a capsule, each unit dosage suitably containing 0.5 to 10 mg of FTY720, e.g. 0.5 mg.

Suitable unit dosage forms for oral administration comprise from ca. 0.1 to 30 mg, usually 0.25 to 30 mg S1P receptor modulator or agonist, together with one or more pharmaceutically acceptable diluents or carriers therefore. The S1P receptor modulator or agonist, e.g. FTY720 or a pharmaceutically acceptable salt thereof, may be administered at a dose of 0.5 mg or 1.25 mg, e.g. 0.5 mg.

Utility of an S1P receptor modulator or agonist in treating diseases and conditions as hereinabove specified may be demonstrated in standard animal or clinical tests, e.g. in accordance with the methods described hereinafter.

### EXAMPLE

Annualized relapse rate (ARR) over 24 months (primary endpoint) and time to disability progression (key secondary endpoint) is assessed in patients with relapsing—remitting MS (RRMS). Patients aged 18-55 years with an Expanded Disability Status Scale score 0-5.5, ≧1 relapse in the previous year (or ≧2 in the previous 2 years) are randomized to daily oral fingolimod 1.25 mg or 0.5 mg, or placebo.

MRI scans are performed at screening and at month 12. The brain volume changes for months 0 to 12 is measured using SIENA software.

### Results

Patients in both fingolimod groups have significantly fewer Gd-enhancing lesions than those in the placebo group at 6, 12 and 24 months, and proportionately more patients are free from Gd-enhancing lesions in both fingolimod groups at these timepoints. Fewer new or enlarged T2 lesions are observed over 24 months with fingolimod 0.5 mg or 1.25 mg, and greater proportions of patients are free from these lesions with both fingolimod doses. The volume of T2 lesions decreases from baseline to month 24 in fingolimod-treated patients and increases with placebo. Furthermore, fingolimod-treated patients have significantly less reduction in brain volume than those in the placebo group from baseline to month 24, i.e. they show a 30% reduction in atrophy rate.

| | fingolimod | | |
|---|---|---|---|
| | 1.25 mg | 0.5 mg | placebo |
| | N = 429 | N = 425 | N = 418 |
| Patients free from | 175 (51.9) | 187 (50.5) | 72 (21.2) |
| new or enlarged | | | |
| T2 lesions, | | | |
| months | | | |
| 0-24 - no. (%) | | | |
| T2 lesion volume, | N = 343^{§§} | N = 368^{§§} | N = 339^{§§} |
| months 0-24 - % | | | |
| change | | | |
| Mean ± SD | 1.6 ± 30.71 | 10.6 ± 103.46 | 33.8 ± 106.90 |
| Median | −3.10 | −1.69 | 8.61 |
| (range) | (−68.2-221.5) | (−100.0-1828.5) | (−84.5-1378.7) |
| Measures of | | | |
| tissue | | | |
| integrity/loss | | | |
| T1 hypointense | N = 317^{§§} | N = 346^{§§} | N = 305^{§§} |
| lesion volume, | | | |
| months 0-24 - | | | |
| % change | | | |
| Mean ± SD | 12.2 ± 85.49 | 8.8 ± 76.27 | 50.7 ± 388.26 |
| Median | −0.20 | 0.00 | 1.59 |
| (range) | (−100.0-888.4) | (−100.0-1037.1) | (−100.0-5285.3) |

^{§§}Number of patients with available magnetic resonance imaging data.

| | FTY720 1.25 mg | FTY720 0.5 mg | Placebo |
|---|---|---|---|
| | N = 429 | N = 425 | N = 418 |
| Percent change from baseline to Month 6 | | | |
| n | 384 | 395 | 383 |
| Mean (SD) | −0.209 (0.8649) | −0.224 (0.8131) | −0.344 (0.7334) |
| Median | −0.120 | −0.140 | −0.290 |
| Range | −4.71 to 3.37 | −5.62 to 2.25 | −4.02 to 2.57 |
| P-value for treatment comparison of FTY720 | 0.003* | 0.006* | — |
| vs. placebo (rank ANCOVA with covariates) | | | |
| Percent change from baseline to Month 12 | | | |
| n | 371 | 383 | 358 |
| Mean (SD) | −0.438 (1.0792) | −0.500 (1.0509) | −0.647 (1.0527) |
| Median | −0.300 | −0.380 | −0.560 |
| Range | −4.91 to 4.34 | −8.11 to 2.40 | −3.89 to 2.78 |
| P-value for treatment comparison of FTY720 | 0.001* | 0.026* | — |
| vs. placebo (rank ANCOVA with covariates) | | | |
| Percent change from baseline to Month 24 | | | |
| n | 334 | 357 | 331 |
| Mean (SD) | −0.885 (1.3857) | −0.843 (1.3120) | −1.306 (1.5000) |
| Median | −0.700 | −0.670 | −0.980 |
| Range | −6.33 to 3.04 | −13.50 to 2.16 | −7.58 to 2.38 |
| P-value for treatment comparison of FTY720 | <0.001* | <0.001* | — |
| vs. placebo (rank ANCOVA with covariates) | | | |
| Percent change from Month 12 to Month 24 | | | |
| n | 327 | 356 | 329 |
| Mean (SD) | −0.423 (0.8284) | −0.370 (0.8073) | −0.669 (1.0723) |
| Median | −0.380 | −0.340 | −0.570 |
| Range | −5.40 to 2.24 | −6.24 to 1.90 | −5.60 to 2.43 |
| P-value for treatment comparison of FTY720 | 0.002* | <0.001* | — |
| vs. placebo (rank ANCOVA with covariates) | | | |

n = the number of patients with non-missing baseline and post-baseline values.
P-values are from rank ANCOVA with covariates of treatment, country, and baseline normalized brain volume.
*Indicates two-sided statistical significance at 0.05 level.

1. A method for inhibiting brain atrophy, or limiting, reducing the progression of brain atrophy in a subject in need thereof, comprising administering to the subject a S1P receptor modulator or agonist, e.g. FTY720 or a pharmaceutically acceptable salt thereof, or Compound B.

2. A method for slowing progression of brain atrophy in a subject in need thereof, comprising administering to the subject a S1P receptor modulator or agonist, e.g. FTY720 or a pharmaceutically acceptable salt thereof, or Compound B.

3. A method of claim 1 or 2 wherein the S1P receptor modulator or agonist comprises a group of formula X 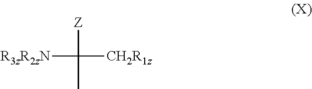
wherein Z is H, C₁₋₆alkyl, C₂₋₆alkenyl, C₂₋₆alkynyl, phenyl, phenyl substituted by OH, C₁₋₆alkyl substituted by 1 to 3 substituents selected from the group consisting of halogen, C₃₋₈cycloalkyl, phenyl and phenyl substituted by OH, or CH₂—R_{4z} wherein R_{4z} is OH, acyloxy or a residue of formula (a) 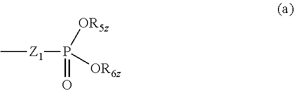
wherein Z₁ is a direct bond or O, preferably O;
each of R_{5z} and R_{6z}, independently, is H, or C₁₋₄alkyl optionally substituted by 1, 2 or 3 halogen atoms;
R_{1Z} is OH, acyloxy or a residue of formula (a); and each of R_{2z} and R_{3z} independently, is H, C₁₋₄alkyl or acyl.

4. A method of claim 1 or 2 wherein the brain atrophy results from an autoimmune disease, e.g. multiple sclerosis.

5. A method of claim 1 or 2 wherein the subject to be treated is affected by an autoimmune disease, e.g. multiple sclerosis.

6. Method according to claim 1 or 2 wherein the S1P receptor modulator or agonist is FTY720, a pharmaceutically acceptable salt or a phosphate thereof, e.g. FTY720 hydrochloride salt.

7. A method of claim 1 or 2 comprising administering to the subject a daily dosage of FTY720 or a pharmaceutically acceptable salt thereof of about 0.5 mg or 1.25 mg.
